(19) United States
(12) Patent Application Publication
(10) Pub. No.: US 2021/0045916 A1
(43) Pub. Date: Feb. 18, 2021

(54) LIQUID MEDICINE ADMINISTRATION IDENTIFYING DEVICE AND LIQUID MEDICINE ADMINISTRATION IDENTIFYING SYSTEM USING SAME

(71) Applicants: SEOUL NATIONAL UNIVERSITY HOSPITAL, Seoul (KR); RESEARCH & BUSINESS FOUNDATION SUNGKYUNKWAN UNIVERSITY, Suwon-si (KR)

(72) Inventors: Tae Woo KIM, Seongnam-si (KR); Tai Myoung CHUNG, Seoul (KR)

(73) Assignees: SEOUL NATIONAL UNIVERSITY HOSPITAL, Seoul (KR); RESEARCH & BUSINESS FOUNDATION SUNGKYUNKWAN UNIVERSITY, Suwon-si (KR)

(21) Appl. No.: 16/966,531

(22) PCT Filed: Feb. 1, 2019
(86) PCT No.: PCT/KR2019/001478

§ 371 Date: Jul. 31, 2020

(30) Foreign Application Priority Data
Feb. 2, 2018 (KR) 10-2018-0013408

(51) Int. Cl. A61F 9/00; A61M 35/00; G16H 20/13

(52) U.S. Cl. CPC A61F 9/0008; A61M 35/00; G16H 20/13; A61M 2205/18; A61M 2205/8206; A61M 2205/582; A61M 2205/583; A61M 2205/3584; A61M 2205/3331; A61M 2205/581

(57) ABSTRACT

A liquid medicine administration identifying device according to an embodiment of the present invention comprises: a body including a receiving space for receiving an administration container, through which a liquid medicine can be administrated; a sensing unit installed at the body to sense that the administration container is compressed or overturned; and a transmission unit for transmitting a sensing result of the sensing unit to the outside.

### TECHNICAL FIELD

The present invention relates to a liquid medicine administration identifying device and a liquid medicine administration identifying system using the same, and more particularly, a liquid medicine administration identifying device for identifying whether or not a liquid medicine is administrated and a liquid medicine administration identifying system using the same.

### BACKGROUND ART

A patient who require continuous administration of an eyewash, such as a glaucoma patient, are subject to considerable stress in remembering a time of eyewash administration, and in particular, in an age group with a lot of social activity, it is common to forget and skip drug administration.

In addition, an elderly patient who have poor memory do not have a clear memory of whether or not drug has been administered, and thus, the elderly patient overdoes the drug or frequently skips the drug.

In addition, during an interview process between the patient and a doctor, in general, the patient's answer is unclear when the doctor asks the patient how the patient faithfully takes the drug. Moreover, when the patient reports to the doctor that the patient takes the drug better than the patient actually takes the drug, in a case where a treatment progress is not good, it is difficult for the doctor to distinguish whether the patient does not faithfully take the drug, and thus, the treatment progress is not improved or whether the treatment progress is not improved even when the patient faithfully takes the drug.

### DISCLOSURE

### Technical Problem

An object of the present invention is to provide a liquid medicine administration identifying device which allows a user and/or a doctor to identify a medication fulfillment state of a liquid medicine, and a liquid medicine administration identifying system using the same.

The objects of the present invention are not limited to above-described object, and other objects not mentioned will be clearly understood by a person skilled in the art from the following description.

### Technical Solution

In order to achieve to the above-described objects, according to an embodiment of the present invention, there is provided a liquid medicine administration identifying device including: a body which includes a receiving space for accommodating an administration container through which a liquid medicine is administrated; a sensing unit which is installed in the body to sense that the administration container is compressed or overturned; and a transmission unit which transmits a sensing result of the sensing unit to an outside.

### Advantageous Effects

Embodiments of the present invention have at least the following effects.

A user and/or a doctor can identify a medication fulfillment state of a liquid medicine.

The effects according to the present invention are not limited by the contents exemplified above, and more various effects are included in the present specification.

### DESCRIPTION OF DRAWINGS

The FIG. 1 is a view schematically illustrating a liquid medicine administration identifying system according to a first embodiment of the present invention.

FIG. 2 is a cross-sectional view schematically illustrating a state in which the liquid medicine administration identifying device and an administration container are coupled with each other according to the first embodiment of the present invention.

FIG. 3 is a block diagram schematically illustrating a configuration of the liquid medicine administration identifying device according to a first embodiment of the present invention.

FIG. 4 is a view illustrating an example of use of a liquid medicine administration identifying device according to the first embodiment of the present invention.

FIGS. 5 and 6 are views for explaining a configuration of a liquid medicine administration identifying device according to a second embodiment of the present invention.

FIGS. 7 and 8 are views for explaining the configuration of the liquid medicine administration identifying device according to the second embodiment.

FIG. 9 is a view schematically illustrating a liquid medicine administration identifying system according to the second embodiment of the present invention.

FIG. 10 is a block diagram schematically illustrating a liquid medicine administration identifying system according to a third embodiment of the present invention.

### MODE FOR DISCLOSURE

Advantages and features of the present invention, and methods for achieving them will be clarified with reference to embodiments described below in detail together with the accompanying drawings. However, the present invention is not limited to the embodiments disclosed below, but may be implemented in various different forms. That is, only the embodiments allow the disclosure of the present invention to be complete and are provided to completely inform the scope of the invention to those skilled in the art to which the present invention pertains, and the present invention is only defined by the scope of the claims. The same reference numerals refer to the same components throughout the specification.

In addition, the embodiments described herein will be described with reference to cross-sectional views and/or schematic drawings, which are ideal exemplary views of the present invention. Therefore, shapes of the exemplary views may be modified by manufacturing technology and/or tolerance. In addition, in each drawing illustrated in the present invention, each component may be illustrated to be slightly enlarged or reduced in consideration of convenience of description. The same reference numerals refer to the same components throughout the specification.

Hereinafter, the present invention will be described with reference to the drawings for describing a liquid medicine administration identifying device and a liquid administration identification system using the same according to embodiments of the present invention.

FIG. 1 is a view schematically illustrating a liquid medicine administration identifying system according to a first embodiment of the present invention.

As illustrated in FIG. 1, a liquid medicine administration identifying system 1 according to the first embodiment of the present invention includes a liquid medicine administration identifying device 10 and a terminal 20.

The terminal 20 is a device capable of wireless communication, and may be a smart phone, a tablet, a computer, or the like, or may be a dedicated terminal manufactured exclusively for the system 1.

As illustrated in FIG. 1, the liquid medicine administration identifying device 10 is coupled with the administration container 30. The administration container 30 may be a general administration container in which a liquid medicine therein is discharged when the container is compressed, such as an eyewash container.

The liquid medicine administration identifying device 10 includes a body 12 in which a receiving space 11 for receiving the administration container 30 is formed. The body 12 may be formed to surround at least a portion of a lower end and a sidewall of the administration container 30. According to an embodiment, the liquid medicine administration identifying device may be formed to surround only a portion of the lower end and the sidewall of the administration container 30, or may be formed to surround only a portion of the sidewall.

When the liquid medicine is discharged while the body 12 comes into close contact with the administration container 30, the body 12 should be compressed and deformed together with the administration container 30. Accordingly, it is preferable that the body 12 is made of a material having excellent ductility and elasticity. For example, the body 12 may be made of a soft polymer material such as silicone.

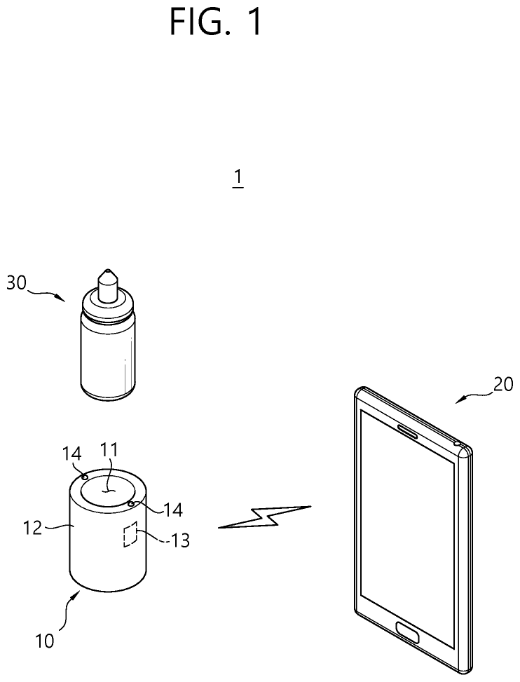

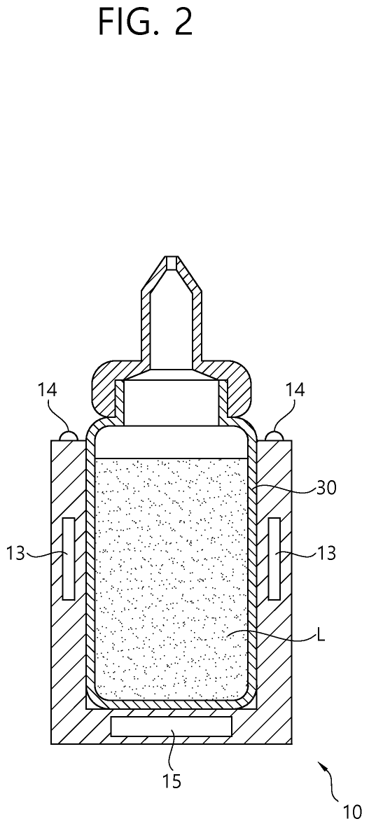

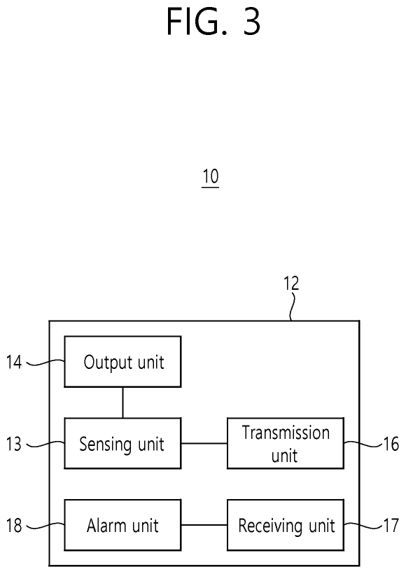

Hereinafter, the liquid medicine administration identifying device 10 will be described in more detail with reference to FIGS. 2 and 3 together with FIG. 1.

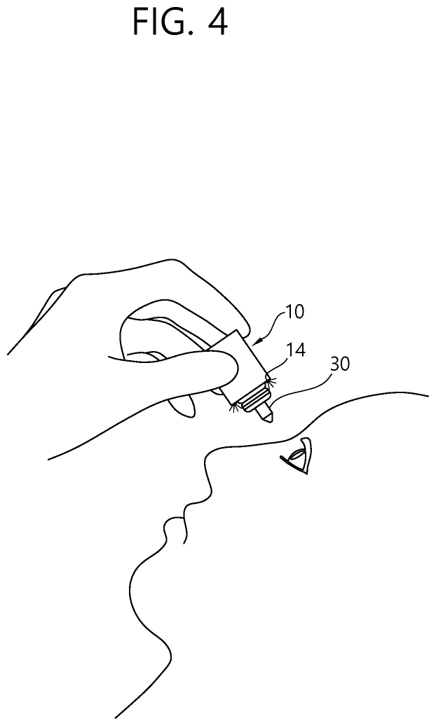

FIG. 2 is a cross-sectional view schematically illustrating a state in which the liquid medicine administration identifying device and the administration container are coupled with each other according to the first embodiment of the present invention, FIG. 3 is a block diagram schematically illustrating a configuration of the liquid medicine administration identifying device according to a first embodiment of the present invention, and FIG. 4 is a view illustrating an example of use of a liquid medicine administration identifying device according to the first embodiment of the present invention.

As illustrated in FIGS. 2 and 3, the liquid medicine administration identifying device 10 according to the present embodiment includes a sensing unit 13, an output unit 14, a transmission unit 16, a receiving unit 17, an alarm unit 18, and a battery 15 in the body 12.

The sensing unit 13 senses that the administration container 30 discharges the liquid medicine. To this end, the sensing unit 13 may be configured to sense that the administration container 30 is compressed.

The sensing unit 13 may include a pressure sensor. The pressure sensor includes all sensors that can sense whether or not compression is performed or a degree of the compression, and for example, includes not only a load cell but also a switch which is turned on/off depending on whether or not the compression is performed.

In a state where the liquid medicine administration identifying device 10 is coupled with the administration container 30, when the user compresses an outer surface of the liquid medicine administration identifying device 10 to take or administer the liquid medicine and the administration container 30 is compressed, the pressure sensor senses an external force which compress the liquid medicine administration identifying device 10 and the administration container 30.

As illustrated in FIG. 2, the sensing unit 13 may be installed inside the body 12, but is not limited thereto. That is, according to an embodiment, the sensing unit 13 may be provided on an inner surface of the body 12 or an outer surface of the body 12 forming a side wall of the receiving space 11, and thus, the sensing unit 13 may be installed at any position as long as the sensing unit 13 can sense the compression of the liquid medicine administration identifying device 10 and the administration container 30.

When the sensing unit 13 senses the external force which compresses the liquid medicine administration identifying device 10 and the administration container 30, the output unit 14 outputs a signal that the user can identify the external force by any one of a visual sense, an auditory sense, and a tactile sense.

As illustrated in FIG. 2, in the present embodiment, as an example of the output unit 14, a light source that outputs light, which is a signal that the user can visually identify, is used.

When the sensing unit 13 senses the external force, the light source of the output unit 14 is turned on. Therefore, the user can visually identify whether or not the sensing unit 13 appropriately senses the external force.

Preferably, the light source is disposed at a position where the user can easily identify whether or not the light source is turned on while the user uses the administration container 30. For example, when a liquid medicine L is an eyewash, as illustrated in FIGS. 2 and 4, a light source may be provided at an upper end of the body 12 facing an eye. Therefore, as illustrated in FIG. 4, the user can visually identify whether or the sensing unit 13 appropriately senses external force by identifying lighting of the light source while the user administers the liquid medicine L to the eye.

When the body 12 is made of a transparent or semi-permeable material, the light source may be provided inside the body 12.

When the sensing unit 13 senses the external force which compresses the liquid medicine administration identifying device 10 and the administration container 30, the transmission unit 16 transmits information that the sensing unit 13 has sensed the external force to the terminal 20.

The transmission unit 16 may transmit the information through wired/wireless communication, but it is preferable that the transmission unit 16 transmits the information through a wireless communication method for user convenience. As a wireless communication method, Wi-Fi, Bluetooth, or the like capable of communicating with the terminal 20 may be used.

An application for managing liquid medicine administration information in conjunction with information transmitted from the liquid medicine administration identifying device 10 is installed in the terminal 20.

The application generates and stores the liquid medicine administration information recorded together with a time when the information transmitted from the transmission unit 16 is received. That is, the application assumes that the administration of liquid medicine has been performed when information indicating that the sensing unit 13 has sensed the external force is transmitted from the transmission unit 16, and records the time when the information is transmitted as an administration time of the liquid medicine.

The user runs the application through the terminal 20 and can identify a record (administration time, number of times, or the like) of liquid medicine administration.

In addition, the doctor can also run the application through the terminal 20 of the user to identify the record (administration time, number of times, or the like) of liquid medicine administration, and identify the medication fulfillment state of the user.

The liquid medicine administration identifying system 1 according to the present embodiment may be configured to allow the user to control operating options of the liquid medicine administration identifying device 10 through the application. For example, the user can adjust an operation option, such as setting brightness, a flashing cycle, or the like of the light source of the output unit 14, or setting a time for providing an alarm through an application.

To this end, the liquid medicine administration identifying device 10 according to the present embodiment includes the receiving unit 17.

The receiving unit 17 receives information transmitted from the terminal 20.

Based on the information received by the receiving unit 17, the output unit 14 controls the brightness, the flashing cycle, or the like of the light source, and the alarm unit 18 provides an alarm at a time set by the user through the application. The alarm may be a signal that the user can identify with any one of the visual sense, the auditory sense, and the tactile sense, and may be provided through the output unit 14 or the alarm unit 18 may separately output an alarm signal.

The battery 15 supplies power required for the operation of the sensing unit 13, the output unit 14, the transmission unit 16, the receiving unit 17, and the alarm unit 18. The battery 15 may be detachably installed from the body 12 and installed to be replaceable, or may be configured to be charged by electric power supplied through a charging port (not illustrated) formed in the body 12.

As illustrated in FIG. 2, preferably, the battery 15 is installed in a lower portion (a portion supporting the lower end of the administration container 30) of the body 12 so that the battery 15 does not disturb the sensing unit 13 sensing that the administration container 30 and the liquid medicine administration identifying device 10 are compressed. However, the installation position of the battery 15 is not limited thereto, and the installation position of the battery 15 may be changed according to an embodiment as long as the sensing of the sensing unit 13 is not disturbed.

Hereinafter, a liquid medicine administration identifying device according to another embodiment will be described. For convenience of description, portions similar to those in the first embodiment use the same reference numerals, and portions common to those in the first embodiment are omitted.

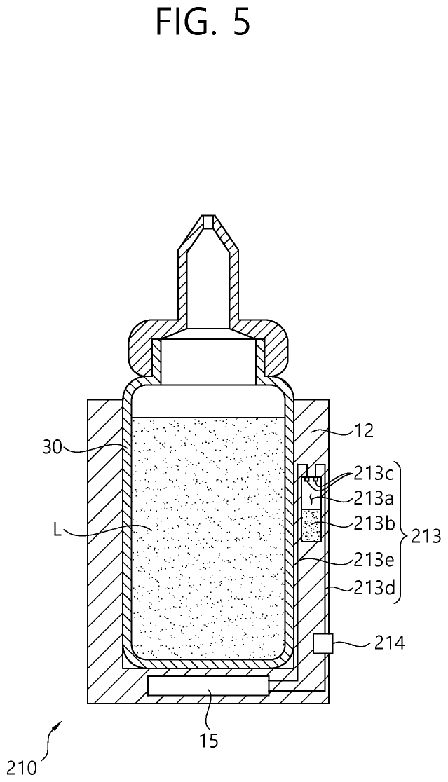

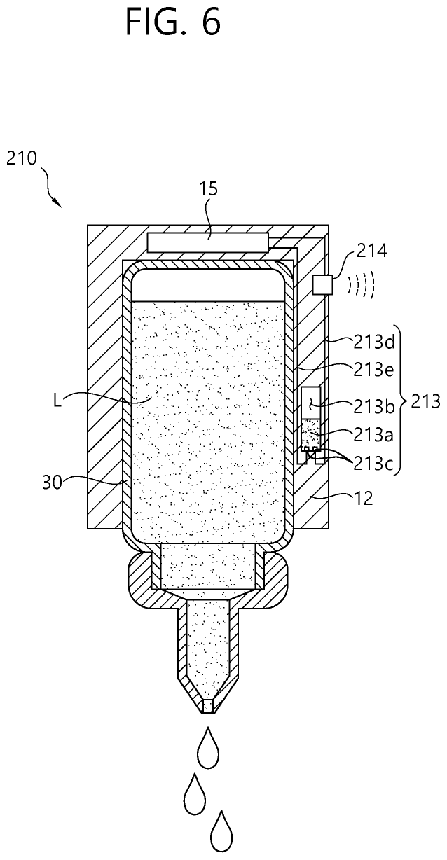

FIGS. 5 and 6 are views for explaining a configuration of a liquid medicine administration identifying device according to a second embodiment of the present invention.

As illustrated in FIG. 5, a liquid medicine administration identifying device 210 according to the second embodiment of the present invention includes a sensing unit 213 which is configured differently from that of the liquid medicine administration identifying device 10 according to the first embodiment described above.

The sensing unit 213 of the liquid medicine administration identifying device 210 according to the present embodiment senses overturn of the liquid medicine administration identifying device 210. In general, when a liquid medicine is administrated, the administration container 30 is overturned. Therefore, in a state where the liquid medicine administration identifying device 210 is coupled with an administration container 30, the sensing unit 213 senses the overturn of the liquid medicine administration identifying device 210 to sense overturn of the administration container 30, and thus, the sensing unit considers this overturn as administration of the liquid medicine.

FIG. 5 illustrates an example of a configuration of the sensing unit 213 capable of sensing the overturn of the liquid medicine administration identifying device 210.

As illustrated in FIG. 5, the sensing unit 213 includes a chamber 213a, a conductive material 213b, a pair of electrodes 213c, and circuits 213d and 213e connected to the battery 15.

The chamber 213a is an enclosed space formed inside the body 12, and preferably, the chamber 231a is formed to have a longer length in an up-down direction than a length in a right-left direction based on FIG. 5.

The conductive material 213b is movably accommodated in the chamber 213a. As the conductive material 213b, a liquid such as water through which a current can be applied, a conductive metal, or the like may be used.

The pair of electrodes 213c is installed to be exposed inside the chamber 213a. The pair of electrodes 213c is installed to be located at a lower portion of the chamber 213a in a state where the liquid medicine administration identifying device 210 is overturned. Therefore, in a normal state where the liquid medicine administration identifying device 210 is not overturned, preferably, the pair of electrodes 213c is located at an upper portion inside the chamber 213a.

The circuits 213d and 213e are formed to connect the pair of electrodes 213c and the battery 15 to each other, and constitute a circuit which is disconnected between the pair of electrodes 213c.

As illustrated in FIG. 5, in the present embodiment, an example including an output unit 214 that outputs a sound which is a signal that a user can identify with an auditory sense is illustrated. The output unit 214 may be connected to the circuits 213d and 213e.

As illustrated in FIG. 5, in the normal state where the liquid medicine administration identifying device 210 is not overturned, the conductive material 213b is located at a lower portion of the chamber 213a due to the gravity, and thus, the conductive chamber 213a does not come into contact with the pair of electrodes 213c. Therefore, a state where the circuits 213d and 213e are disconnected to each other is maintained.

However, as illustrated in FIG. 6, when the liquid medicine administration identifying device 210 is overturned, the conductive material 213b comes into contact with the pair of electrodes 213c by the gravity, and a current can be applied through the circuits 213d and 213e. At the same time, a current is supplied to the output unit 214 to output a sound. That is, the sensing unit 213 according to the present embodiment senses the overturn of the liquid medicine administration identifying device 210 by the application of current through circuits 213d and 213e, and considers that the administration container 30 coupled with the liquid medicine administration identifying device 210 is overturned and the liquid medicine is administered.

When the current is applied to the transmission unit 16 through the circuits 213d and 213e, the transmission unit 16 transmits information on the application of the current to the terminal 20. The user listens to the sound output through the output unit 214, and can audibly identify that the sensing unit 213 senses the overturn of the liquid medicine administration identifying device 210.

In the present embodiment, as an example of the output unit 214, the output unit which outputs the sound is illustrated and described as an example. However, similar to the liquid medicine administration identifying device 10 according to the first embodiment described above, the output unit of outputting light may be also used.

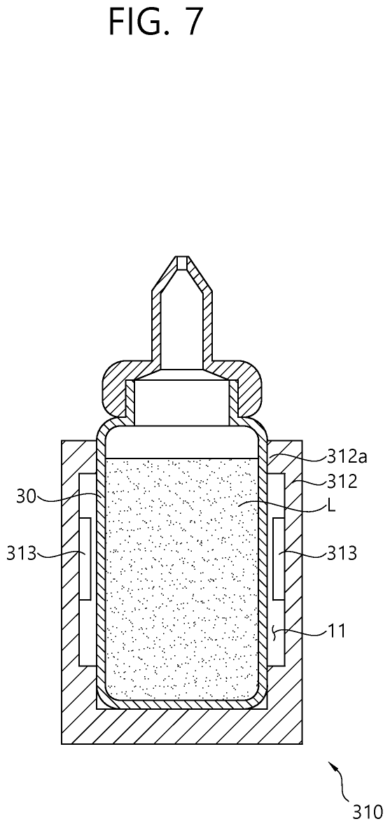

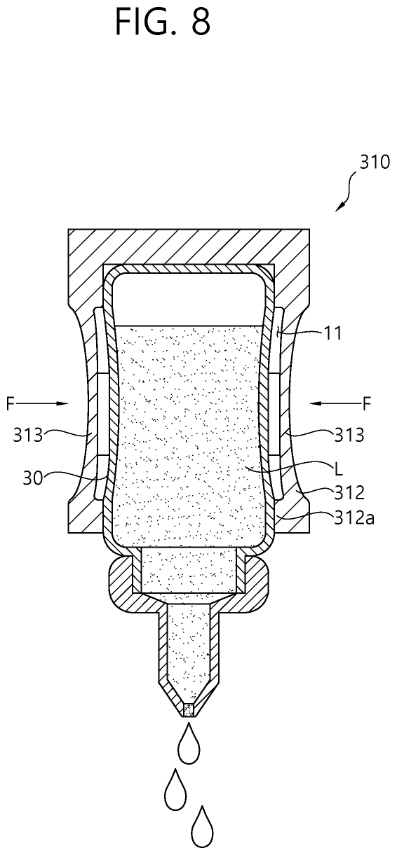

FIGS. 7 and 8 are views for explaining the configuration of the liquid medicine administration identifying device according to the second embodiment.

As illustrated in FIG. 7, a liquid medicine administration identifying device 310 according to the third embodiment of the present invention includes a body 312 and a sensing unit 313 having configurations different from those of the liquid medicine administration identifying device 10 according to the first embodiment described above.

The sensing unit 313 of the liquid medicine administration identifying device 310 according to the present embodiment senses that the body 312 of the liquid medicine administration identifying device 310 is compressed toward the administration container 30.

To this end, the sensing unit 313 includes a switch located between the body 312 and the administration container 30 in a state where the liquid medicine administration identifying device 310 is coupled with the administration container 30.

As illustrated in FIG. 7, the switch is not effectively pressed in a state where the liquid medicine administration identifying device 310 is not compressed toward the administration container 30. However, as illustrated in FIG. 8, the switch is effectively pressed by the administration container 30 in a state where the liquid medicine administration identifying device 310 is compressed toward the administration container 30 by an external force F.

To this end, the body 312 is formed so that an upper end 312a protrudes toward a receiving space 11 to support a side portion of the administration container 30, and in the state where the liquid medicine administration identifying device 310 is coupled with the administration container 30, a gap may be formed between a portion an inner side wall of the receiving space 11 from which the switch is exposed and the administration container 30.

When the switch is effectively pressed between the administration container 30 and the body 312, the transmission unit 16 transmits information on the pressing to the terminal 20. To this end, the switch may be configured to generate an electrical signal in a state of being effectively pressed and transmit the electric signal to the transmission unit 16.

In addition, the switch is configured to generate a click sensation that the user can sense the pressing of the switch by a tactile sense when the switch is effectively pressed. Accordingly, the user can tactilely identify that the sensing unit 313 senses the external force in a state where the user applies the external force F to administrate the liquid medicine.

Alternatively, an output unit which generates vibration when the switch is effectively pressed may be provided so that the user may tactilely identify that the sensing unit 313 senses the external force.

In the present embodiment, as an example of the output unit, a configuration that the user can recognize through the tactile sense is described. However, similar to the liquid medicine administration identifying devices 10 and 210 according to the first or second embodiments described above, the output unit which outputs light or a sound can be used.

Hereinafter, a liquid medicine administration identifying system according to another embodiment will be described. For convenience of description, portions similar to those in the first embodiment use the same reference numerals, and portions common to those in the first embodiment are omitted.

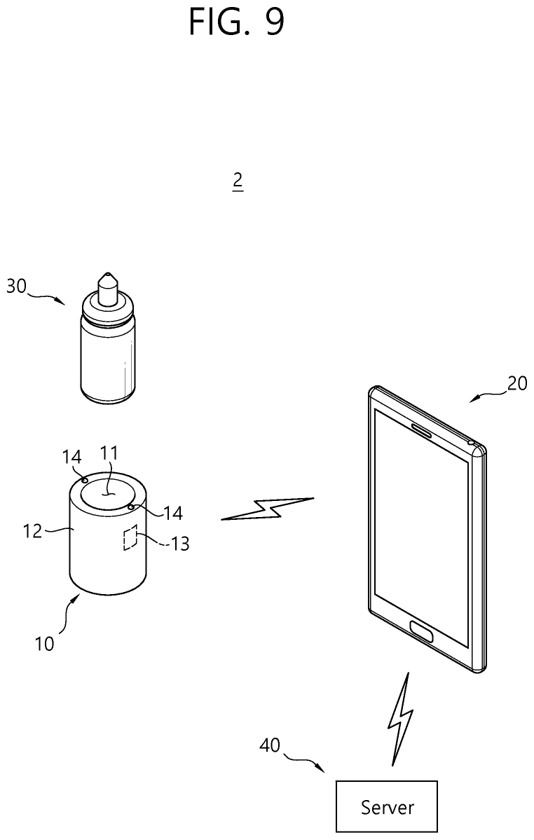

FIG. 9 is a view schematically illustrating a liquid medicine administration identifying system according to a second embodiment of the present invention.

As illustrated in FIG. 9, compared to the liquid medicine administration identifying system 1 according to the first embodiment described above, a liquid medicine administration identifying system 2 according to the second embodiment of the present invention further includes a server 40.

The server 40 is configured to enable wired communication and/or wireless communication with the terminal 20, and the terminal 20 transmits liquid medicine administration information to the server 40 side through an application.

The server 40 stores the transmitted liquid medicine administration information, and upon request, may transmit liquid medicine administration information to a PC, a terminal, or the like of the doctor connected to the server 40.

The server 40 may store and manage liquid medicine administration information of a plurality of different users. The server 40 may receive liquid medicine administration information from a plurality of terminals 20 in which an application is installed, and may classify and store liquid medicine administration information according to identification information of each user.

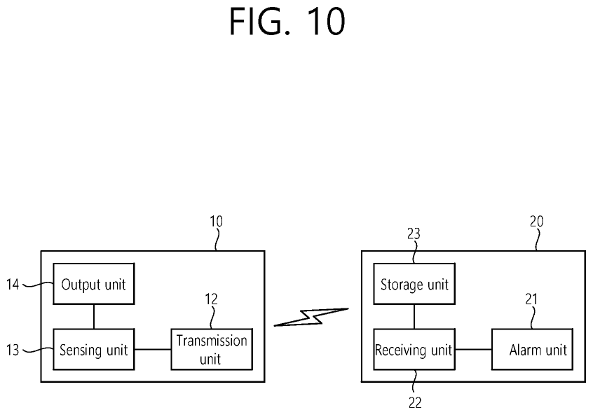

FIG. 10 is a block diagram schematically illustrating a liquid medicine administration identifying system according to a third embodiment of the present invention.

As illustrated in FIG. 10, compared to the liquid medicine administration identifying system 1 according to the first embodiment described above, in a liquid medicine administration identifying system 3 according to the third embodiment of the present invention, the liquid medicine administration identifying device 10 does not include the receiving unit and the alarm unit.

The liquid medicine administration identifying system 3 according to the third embodiment of the present invention uses an alarm unit 21 of the terminal 20.

In the liquid medicine administration identifying system 3 according to the third embodiment of the present invention, the user sets an alarm to output the alarm at a time of administration through the alarm unit 21 of the terminal 20.

When the terminal 20 is a general-purpose terminal such as a smart phone, a tablet, or a computer, the alarm can be set by an alarm function built into the general-purpose terminal, and in a case where the terminal 20 is a dedicated terminal of the system 3, when the user inputs an alarm time, the terminal 20 may include an alarm function of outputting an alarm at the alarm time.

When the alarm is output at the time set by the user by the alarm unit 21 of the terminal 20 (or the time becomes a time when the alarm is set), the receiving unit 22 of the terminal 20 is in a reception waiting state of receiving a signal transmitted from the liquid medicine administration identifying device 10. In this case, as the application installed in the terminal 20 is activated, the receiving unit 22 of the terminal 20 may be in the reception waiting state.

The user recognizes that the time of administration has been reached through the alarm output from the terminal 20, and can perform the administration using liquid medicine administration identifying device 10. As the user performs the administration using the liquid medicine administration identifying device 10, the sensing unit 13 of the liquid medicine administration identifying device 10 senses the administration, and the transmission unit 12 transmits the sensed signal transmitted from the sensing unit 13 to the terminal 20.

When the receiving unit 22 of the terminal 20 receives the signal transmitted from the transmission unit 12, the alarm unit 21 does not output the alarm until the next alarm time, and the storage unit 23 records a time when the receiving unit 22 receives the signal as the administration time of the liquid medicine.

Meanwhile, when a signal is not received through the receiving unit 22 of the terminal 20 for a certain period of time after the alarm is output from the terminal 20, the user considers that the medicine has not been administered, and the alarm unit 21 may output the alarm again.

The number of times the alarm unit 21 outputs the alarm again and/or a time to wait until the alarm is output again may be set in advance or may be changed by the user.

Those of ordinary skill in the art to which the present invention pertains will appreciate that the present invention may be implemented in other specific forms without changing a technical spirit or an essential feature of the present invention. Therefore, it should be understood that the embodiments described above are illustrative in all respects and not restrictive. A scope of the present invention is indicated by the following claims rather than the above detailed description, and it should be interpreted that all changes or modified forms derived from the meaning and scope of claims and equivalent concepts thereof are included in the scope of the present invention.

According to an embodiment of the present invention, there is provided a liquid medicine administration identifying device including: a body which includes a receiving space for accommodating an administration container through which a liquid medicine is administrated; a sensing unit which is installed in the body to sense that the administration container is compressed or overturned; and a transmission unit which transmits a sensing result of the sensing unit to an outside.

The sensing unit may be a pressure sensor which senses that the administration container is compressed.

The sensing unit may include a switch, and the switch may be operated as the body is compressed to the administration container side and a distance between at least a portion of the body and the administration container is changed.

The sensing unit may include a conductive material, a chamber which movably accommodates the conductive material, and an electrode which is exposed to inside an upper side of the chamber, and when the body coupled with the administration container is overturned together with the administration container, the conductive material may come into contact with the electrode so that a current is applied to the conductive material.

The liquid medicine administration may further include an output unit which outputs a signal that a user identifies by any one of a visual sense, an auditory sense and a tactile sense according to the sensing result of the sensing unit.

The liquid medicine administration identifying device may further include an alarm unit which provides an alarm at a set time.

According to another embodiment of the present invention, there is provided a liquid medicine administration identifying system including: a terminal which performs wireless communication; and a medicine administration identifying device which performs wireless communication with the terminal, in which the medicine administration identifying device includes a body which includes a receiving space for accommodating an administration container through which a liquid medicine is administrated, a sensing unit which is installed in the body to sense that the administration container is compressed or overturned, and a transmission unit which transmits a sensing result of the sensing unit to the terminal through wireless communication.

The liquid medicine administration identifying system may further include an application which is installed in the terminal, in which the application may generate liquid medicine administration information which is recorded together with a time when the application receives the sensing result of the sensing unit received from the transmission unit of the medicine administration identifying device.

The liquid medicine administration identifying system may further include a server which communicates with the terminal, in which the application may transmit the liquid medicine administration information to the server, and the server may store the liquid medicine administration information transmitted from the application.

The medicine administration identifying device may further include an alarm unit which provides an alarm, and when a user sets a time when the alarm is provided through the application, the alarm unit may provide the alarm at a set time.

The terminal may further include an alarm unit which provides an alarm at a set time, and when the terminal does not receive the sensing result of the sensing unit for a certain period of time after the alarm unit provides an alarm, the alarm unit may provide an alarm again.

1. A liquid medicine administration identifying device comprising:
a body which includes a receiving space for accommodating an administration container through which a liquid medicine is administrated;
a sensing unit which is installed in the body to sense that the administration container is compressed or overturned; and
a transmission unit which transmits a sensing result of the sensing unit to an outside.

2. The liquid medicine administration identifying device of claim 1, wherein the sensing unit is a pressure sensor which senses that the administration container is compressed.

3. The liquid medicine administration identifying device of claim 1, wherein the sensing unit includes a switch, and
the switch is operated as the body is compressed to the administration container side and a distance between at least a portion of the body and the administration container is changed.

4. The liquid medicine administration identifying device of claim 1, wherein the sensing unit includes a conductive material,
a chamber which movably accommodates the conductive material, and
an electrode which is exposed to inside an upper side of the chamber, and
when the body coupled with the administration container is overturned together with the administration container, the conductive material comes into contact with the electrode so that a current is applied to the conductive material.

5. The liquid medicine administration identifying device of claim 1, further comprising:
an output unit which outputs a signal that a user identifies by any one of a visual sense, an auditory sense and a tactile sense according to the sensing result of the sensing unit.

6. The liquid medicine administration identifying device of claim 1, further comprising:
an alarm unit which provides an alarm at a set time.

7. A liquid medicine administration identifying system comprising:
a terminal which performs wireless communication; and
a medicine administration identifying device which performs wireless communication with the terminal,
wherein the medicine administration identifying device includes
a body which includes a receiving space for accommodating an administration container through which a liquid medicine is administrated,
a sensing unit which is installed in the body to sense that the administration container is compressed or overturned, and
a transmission unit which transmits a sensing result of the sensing unit to the terminal through wireless communication.

8. The liquid medicine administration identifying system of claim 7, further comprising:
an application which is installed in the terminal,
wherein the application generates liquid medicine administration information which is recorded together with a time when the application receives the sensing result of the sensing unit received from the transmission unit of the medicine administration identifying device.

9. The liquid medicine administration identifying system of claim 8, further comprising:
a server which communicates with the terminal,
wherein the application transmits the liquid medicine administration information to the server, and the server stores the liquid medicine administration information transmitted from the application.

10. The liquid medicine administration identifying system of claim 8, wherein the medicine administration identifying device further includes an alarm unit which provides an alarm, and
when a user sets a time when the alarm is provided through the application, the alarm unit provides the alarm at a set time.

11. The liquid medicine administration identifying system of claim 7, wherein the terminal further includes an alarm unit which provides an alarm at a set time, and
when the terminal does not receive the sensing result of the sensing unit for a certain period of time after the alarm unit provides an alarm, the alarm unit provides an alarm again.
